(19) United States
(12) Patent Application Publication
(10) Pub. No.: US 2026/0157978 A1
(43) Pub. Date: Jun. 11, 2026

(54) PROTRUSION PATCH AND METHOD FOR MANUFACTURING SAME

(71) Applicants: HIKS CNT CO., LTD., Seongnam-si, Gyeonggi-do (KR); Kwang Su KIM, Seoul (KR)

(72) Inventor: Kwang Su KIM, Seoul (KR)

(73) Assignees: HIKS CNT CO., LTD., Seongnam-si, Gyeonggi-do (KR); Kwang Su KIM, Seoul (KR)

(21) Appl. No.: 19/127,217

(22) PCT Filed: Nov. 3, 2023
(86) PCT No.: PCT/KR2023/017593

§ 371 Date: May 5, 2025

(30) Foreign Application Priority Data
Nov. 7, 2022 (KR) 10-2022-0147222

(51) Int. Cl. A61K 9/70; A61K 31/728

(52) U.S. Cl. CPC A61K 9/7084; A61K 31/728

(57) ABSTRACT

A protrusion patch includes an adhesive layer and multiple patterned protrusions spaced apart from each other and attached to one surface of the adhesive layer. In the protrusion patch, the patterned protrusions contain a drug and a drug carrier, and the protrusions have a solvent content of 10% to 11% by weight. A method for manufacturing the protrusion patch.

### TECHNICAL FIELD

The present invention relates to a protrusion patch and a method for manufacturing the same.

### BACKGROUND ART

The skin is considered the largest organ in the body with a surface area of about 1.5 m² in adults and may be a preferred route of administration for drugs. However, the stratum corneum, the outermost layer of the skin, which protects the human body from toxic chemicals, makes it difficult for high molecular weight and hydrophilic molecules to pass through this membrane. Among various approaches to enhance transdermal drug delivery, the use of protrusions as a non-invasive method is a promising approach to increase the permeability of the skin to drugs.

Unlike subcutaneous needles, the protrusions allow a painless and non-invasive method of drug administration, are safe after use, and have low disposal costs.

The protrusions create microchannels within the skin, allowing drug molecules to be easily transported through them into the dermis. Several studies have shown that intradermal administration of certain drugs using protrusions increases therapeutic efficacy compared to intramuscular and subcutaneous routes. Thus, the protrusions may provide a route for transdermal drug delivery for molecules that are not readily absorbed locally.

The protrusions are usually made using a molding method, and the molds are manufactured using complex micromachining procedures in a clean room. These procedures include photolithography using deep X-ray lithography and ultraviolet (UV) lithography in LIGA (Lithographie, Galvanoformung, Abformung). However, these methods are very time-consuming, require advanced clean room facilities, and increase manufacturing costs excessively.

In addition, the protrusions are usually formed in an acupuncture shape, and these types of needles have a small contact area with the skin, and thus, they cannot effectively and quickly deliver drugs, and in particular, when applied to a wound, they cause the problem of irritating the wound and inhibiting the therapeutic effect.

### PRIOR ART REFERENCES

### Patent Documents

Korean Patent No. 10-2369762

### DISCLOSURE

### Technical Problem

The present invention has been devised to solve the above problems of the prior arts.

It is an object of the present invention to provide a protrusion patch that can increase the delivery speed of a drug by greatly increasing the contact area between protrusions and the skin, effectively deliver a drug through a wide area without irritating a wound when patched on the wound, stably attach the patch to the skin by increasing the contact area between the skin and an adhesive layer, and greatly lower the manufacturing cost by remarkably reducing the number of manufacturing processes; and a method for manufacturing the same.

In addition, it is another object of the present invention to provide a protrusion patch that has excellent productivity and quality by preventing the protrusions from being detached from the adhesive layer when punched; and a method for manufacturing the same.

### Technical Solution

In order to achieve the above objects, the present invention provides a protrusion patch including:
an adhesive layer; and
multiple patterned protrusions spaced apart from each other and attached to one surface of the adhesive layer,
wherein the patterned protrusions contain a drug and a drug carrier, and
the protrusions have a solvent content of 10% to 11% by weight.

In addition, the present invention provides a method for manufacturing the protrusion patch of the present invention, including the steps of:
a) laminating a protrusion-forming sheet on one surface of an adhesive sheet;
b) punching multiple patterned protrusions into the protrusion-forming sheet; and
c) removing the protrusion-forming sheet except for the protrusions patterned by the punching from the adhesive sheet,
wherein the patterned protrusions contain a drug and a drug carrier, and
the protrusion-forming sheet has a solvent content of 10% to 11% by weight.

In addition, the present invention provides a method for manufacturing the protrusion patch of the present invention, including the steps of:
a) laminating a protrusion-forming sheet on one surface of a first adhesive sheet;
b) punching multiple patterned protrusions into the protrusion-forming sheet;
c) removing the protrusion-forming sheet except for the protrusions patterned by the punching from the first adhesive sheet,
d) laminating a second adhesive sheet on the upper surface of the patterned protrusions; and
e) separating the second adhesive sheet and the protrusions adhered thereto from the first adhesive sheet,
wherein the patterned protrusions contain a drug and a drug carrier, and
the protrusion-forming sheet has a solvent content of 10% to 11% by weight.

### Advantageous Effects

By including patterned protrusions, the protrusion patch of the present invention provides the effects of:
increasing the delivery speed of a drug by greatly increasing the contact area between protrusions and the skin,
effectively delivering a drug through a wide area without irritating a wound unlike conventional acupuncture needles when patched on the wound, and
remarkably reducing the number of manufacturing processes since the protrusions are easily formed by a punching process, thereby manufacturing at a low cost.

In addition, since the patterned protrusion is directly bonded to an adhesive layer without separate support, it provides the effect of remarkably increasing the area of the adhesive layer in contact with the skin, compared to a conventional patch using a separate support.

In addition, the method for manufacturing the protrusion patch of the present invention is very simple and provides high productivity compared to conventional methods. In addition, it provides the effect of remarkably reducing the manufacturing cost of the protrusion patch due to these effects.

In addition, the present invention provides a protrusion patch that has excellent productivity and quality by preventing the protrusions from being detached from the adhesive layer when punched; and a method for manufacturing the same.

### DESCRIPTION OF DRAWINGS

FIGS. 1 and 2 are cross-sectional views schematically showing one embodiment of the protrusion patch of the present invention.

FIGS. 3 and 4 are drawings schematically showing one embodiment of a method for manufacturing the protrusion patch of the present invention.

FIGS. 5 and 6 are drawings schematically showing another embodiment of a method for manufacturing the protrusion patch of the present invention.

FIG. 7 is a photograph of the protrusion patch manufactured in Example 1 of the present invention.

FIG. 8 is a photograph of the protrusion patch manufactured in Example 2 of the present invention.

FIG. 9 is a photograph of the protrusion patch manufactured in Comparative Example 1.

FIG. 10 is a photograph of the protrusion patch manufactured in Comparative Example 2.

### BEST MODE

Hereinafter, the embodiment of the present invention will be described in detail with reference to the accompanying drawings so that a person having ordinary skill in the art to which the present invention pertains can easily practice the invention. However, the present invention may be embodied in many different forms and is not limited to the embodiment set forth herein. Similar parts are designated by the same drawing reference numerals throughout the specification.

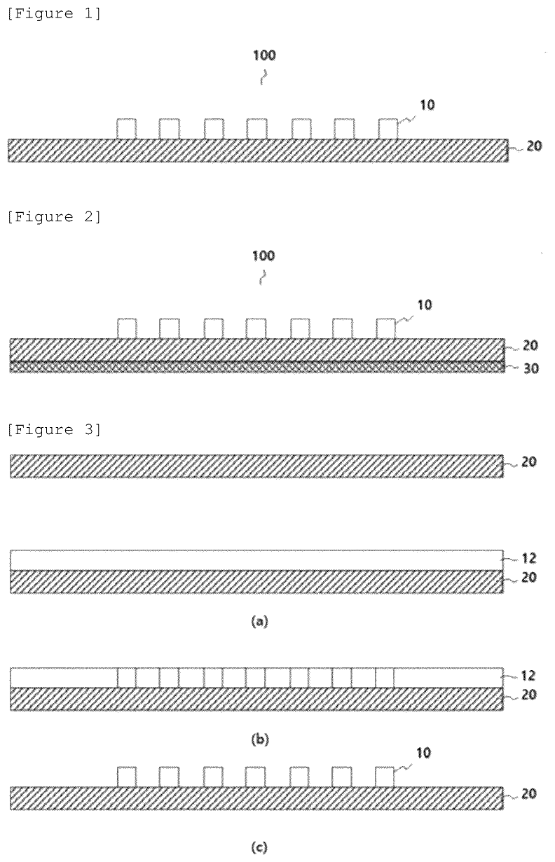

FIGS. 1 and 2 are cross-sectional views schematically showing one embodiment of the protrusion patch according to the present invention. As shown in FIG. 1, the protrusion patch 100 of the present invention includes an adhesive layer 20 and multiple patterned protrusions 10 spaced apart from each other and attached to one surface of the adhesive layer 20, wherein the patterned protrusions 10 contain a drug and a drug carrier, and the protrusions may contain 10% to 11% by weight of a solvent based on the total weight of the protrusions. When the solvent is contained in the above range, since it is prevented that the protrusions are not fixed to the adhesive layer and are detached therefrom when punched to result in the production of defective products, excellent production efficiency and quality may be provided.

In this case, if the solvent is less than 10% by weight, the protrusions have insufficient adhesion to the adhesive layer and may easily be detached from the adhesive layer when punched, and if it exceeds 11% by weight, a problem may occur in which the protrusions stick to the punching apparatus and are detached from the adhesive layer.

As the solvent, one or more selected from water (e.g., distilled water), lower alcohol, glycerin, and the like may be used, and water may be preferably used. Distilled water may preferably be used as the water.

In the protrusion patch of the present invention, the patterned protrusions 10 is directly attached to the adhesive layer 20 without a support, and thus the adhesive layer covered by the support in the conventional patch is revealed and the patch adhesion is further improved. That is, there is an adhesive surface not only at both ends of the patch but also at the central part, and accordingly, the area of the adhesive layer attached to the skin is dramatically expanded compared to the prior art of forming protrusions on a support. As the area of the adhesive layer increases in this way, it is possible for the protrusions 10 to maintain a more solid attachment to the skin.

In addition, for this reason, even if the surface to be attached is curved or the subject to be attached moves, a much better attachment may be maintained compared to the prior art.

In one embodiment of the present invention, the patterned protrusions may have a maximum width 0.1 mm to 50 mm, preferably 1 mm to 10 mm, and a height of 0.01 mm to 1 mm, preferably 0.01 mm to 0.1 mm.

The protrusions may be formed in the shape of a cylinder, an elliptical column or a polygonal column. If the protrusions are formed in the shape of a polygonal column as described above, the surface in contact with the skin becomes a flat shape, and thus, the contact area between the protrusions and the skin is greatly increased, and accordingly, the delivery speed of a drug may be greatly improved. In addition, when the protrusion patch is patched on a wound, the drug may be effectively delivered through a wide area without irritating the wound unlike conventional acupuncture needles. For example, if hyaluronic acid, agents for preventing or treating acne, and the like are contained in the protrusions, the drugs may be effectively delivered through a wide contact area, and thus, excellent therapeutic effects may be provided. In addition, in the case of agents for treating acne, the drugs may be effectively delivered without irritating acne.

In addition, if the protrusions are formed in the shape of a polygonal column as described above, the protrusions may be easily formed by a punching process, and accordingly, the number of manufacturing processes may be greatly reduced. Therefore, the manufacturing unit cost of the protrusion patch may be remarkably reduced by improving productivity.

In one embodiment of the present invention, the protrusion patch 100 may include multiple protrusions arranged at uniform intervals. In addition, the protrusion patch may include one or more groups of protrusions positioned at a distance from each other, and the protrusions included in each group may be arranged to have a uniform interval.

In one embodiment of the present invention, a supporting substrate 30 may be further provided on the other surface of the adhesive layer, as shown in FIG. 2. In addition, an upper cover (not shown) may be further provided on the upper part of the adhesive layer to which the protrusions are attached, and as the supporting substrate 30 and the upper cover, those known in the art may be used without limitation. For example, a polyurethane film may be used as the supporting substrate 30, and a release film may be used as the upper cover.

Hereinafter, the composition of the adhesive layer and protrusions of the present invention will be described in more detail.

### Adhesive Layer

In one embodiment of the present invention, the adhesive layer 20 may be manufactured from a material known in the art, and the material is not particularly limited.

The adhesive layer 20 may include a synthetic rubber component. The synthetic rubber may include, for example, styrene-isoprene Copolymer, polydimethylsiloxane, butyl rubber, ethylene-vinylacetate copolymer, ethylene-ethylacrylate copolymer, poly(alkyl vinyl ether) (e.g., poly(propyl vinyl ether), poly(isopropyl vinyl ether), poly(butyl vinyl ether), etc. ), poly(2-methylpropene), poly(ethylethylene), poly(1,2-dimethylethylene), ethylethylene-1,2-dimethylethylene copolymer, polyisoprene, polybutadiene, styrene-isoprene-styrene block copolymer, styrene-butadiene-styrene block copolymer, etc., and these may be used alone or in a combination of two or more thereof.

The content of the synthetic rubber is not particularly limited, but may be 5% to 50% by weight, preferably 10% to 30% by weight, and more preferably 15% to 25% by weight, based on the total weight of the adhesive layer. If the content of the synthetic rubber is less than 5% by weight, there is a risk that the internal cohesiveness of the adhesive layer may be reduced, and if it is more than 50% by weight, there is a risk that the adhesive layer becomes hard and the adhesiveness is reduced.

The adhesive layer 20 may include a tackifier. The tackifier is not particularly limited, but may include, for example, polybutenes, rosin-based resins, terpene-based resins, petroleum-based resins [e.g., petroleum-based aliphatic hydrocarbon resins, petroleum-based aromatic hydrocarbon resins, petroleum-based aliphatic/aromatic copolymer hydrocarbon resins, petroleum-based alicyclic hydrocarbon resins (hydrogenated aromatic hydrocarbon resins), etc.], coumarone-based resins, etc. Among them, petroleum-based alicyclic hydrocarbon resins (hydrogenated aromatic hydrocarbon resins), for example, hydrogenated hydrocarbon (C=6-20) polymers (Hydrogenated Poly (C6-20 Olefin) ) are preferable. The tackifiers may be used alone or in a combination of two or more thereof.

The tackifier may be included in an amount of 20% to 50% by weight, preferably 35% to 45% by weight, based on the total weight of the adhesive layer. If the content of the tackifier is less than 20% by weight, the adhesiveness may be insufficient, and if it exceeds 50% by weight, it is not preferable because the adhesive layer may be destroyed.

The adhesive layer 20 may include carboxymethyl cellulose. The carboxymethyl cellulose may be included in an amount of 20% to 45% by weight, preferably 25% to 32% by weight, based on the total weight of the adhesive layer.

The adhesive layer 20 may include oils. As the oils, any component known in the art may be used, and for example, mineral oil may be preferably used. The oils may be included in an amount of 5% to 20% by weight, more preferably 8% to 13% by weight, based on the total weight of the adhesive layer.

The adhesive layer 20 may further include an additive commonly used in the art in addition to the components. The additive may be included in an amount of 1% to 10% by weight based on the total weight of the adhesive layer.

The adhesive layer of the present invention may be manufactured by applying and drying an adhesive layer-forming composition onto a release liner to form an adhesive layer, and then laminating a supporting substrate on this adhesive layer. In addition, the adhesive layer may also be manufactured on a support by directly applying and drying the adhesive layer-forming composition onto the supporting substrate.

### Patterned Protrusion

The patterned protrusions may release the drug by dissolving or gelling at a certain rate when contact with the skin.

The protrusions may contain a drug, a drug carrier and a solvent.

As the drug, a drug that can be administered transdermally may be used. Examples may include general anesthetics, hypnotic sedative agents, antiepileptic agents, antipyretic analgesic antiphlogistic agents, antidinics, psychotropic agents, agents acting on central nervous system, antidementia drugs, local anesthetics, skeletal muscle relaxants, agents acting on autonomic nervous system, antispasmodic agents, antiparkinson drugs, antihistaminic agents, cardiac stimulants, antiarrhythmic agents, diuretics, antihypertensive agents, vasoconstrictors, vasodilators, peripheral vasodilators, antiarteriosclerotic agents, circulatory agents, respiratory stimulators, antitussive agent and expectorants, hormone drugs, medicines for external application for treating purulent diseases, analgesic, antipruritic, astringent and antiphlogistic agents, drugs against parasitic skin diseases, styptics, agents for gout, antidiabetics, anti-malignant tumor agents, antibiotics, agents for chemotherapy, narcotics, hyaluronic acid, agents for preventing and treating acne, drugs used in nicotine dependence, etc.

In addition, the drug includes not only the drug as a free base, but also its physiologically acceptable salts. The salts are not particularly limited, but may include, for example, formate, acetate, lactate, adipate, citrate, tartrate, methanesulfonate, fumarate, maleate, etc., and as addition salts of inorganic acids, hydrochloride, sulfate, nitrate, phosphate, and the like may be exemplified. In addition, the drug may be a solvate, a hydrate or a non-hydrate.

Specifically, hyaluronic acid, agents for preventing and treating acne, and the like may be used.

As the drug carrier, a component that may be gelled by a solvent released from the skin and may release the drug contained in the drug carrier due to gelation of the drug-supporting layer may be used. For example, one alone or a combination of two or more selected from the group consisting of hyaluronic acid, polyvinylpyrrolidone (PVP), polyvinyl alcohol (PVA), sodium carboxymethyl cellulose (NaCMC), poloxamer, carbomer, hypromellose, hydroxypropyl cellulose (HPC), hydroxyethyl cellulose (HEC), sodium alginate, saccharides, glycerin, propylene glycol, polyethylene glycol 400, sorbitol (SB), and the like may be used.

The saccharides may be one or more selected from the group consisting of monosaccharides, disaccharides and polysaccharides, and more specifically, the monosaccharides may be one more selected from the group consisting of fructose, galactose, glucose and mannose; the disaccharides may be one ore more selected from the group consisting of sucrose, lactose, maltose, trehalose, turanose and cellobiose; and the polysaccharides may be one ore more selected from the group consisting of dextran, diethylaminoethyl dextran, dextrin, cellulose and β-glucans.

As the solvent, water (e.g., distilled water), lower alcohol, glycerin, and the like may be used, and water may be preferably used. Distilled water may preferably be used as the water.

The protrusions may contain 0.1% to 20% by weight of the drug, 69% to 89% by weight of the drug carrier, and 10% to 11% by weight of the solvent. In addition, the protrusions may further contain 0.1% to 10% by weight of functional additives and/or general additives known in the art. In this case, water (e.g., distilled water) may be preferably used as the solvent.

Although the structure of the protrusion patch of the present invention has been described above, it should not be understood that the protrusion patch according to the present invention includes only the components described above. Although not described in the present specification, it should be understood that components that are obvious to a person having ordinary skill in the art may be additionally included in the protrusion patch of the present invention.

Hereinafter, a method for manufacturing the protrusion patch of the present invention will be described by way of examples.

As shown in FIG. 3, the protrusion patch of the present invention may be manufactured by including the steps of:
a) laminating a protrusion-forming sheet 12 on one surface of an adhesive sheet 20;
b) punching multiple patterned protrusions into the protrusion-forming sheet 12; and
c) removing the protrusion-forming sheet 12 except for the protrusions patterned by the punching from the adhesive sheet 20.

The patterned protrusions contain a drug and a drug carrier, and
the protrusion-forming sheet may have a solvent content of 10% to 11% by weight.

Since all of the contents described in the protrusion patch may be applied to all manufacturing methods of the present invention, any duplicate contents will be omitted below.

In addition, the components of the protrusion-forming sheet are the same as those of the protrusion described above.

The method for manufacturing the protrusion patch of the present invention has the characteristic of remarkably reducing the number of manufacturing processes compared to the prior art, and thus, the manufacturing cost of the protrusion patch may be remarkably reduced.

The protrusion-forming sheet may be manufactured in a sheet shape by a method known in the art.

In one embodiment of the present invention, a supporting substrate 30 may be further attached to the other surface of the adhesive sheet before step a) or after step c).

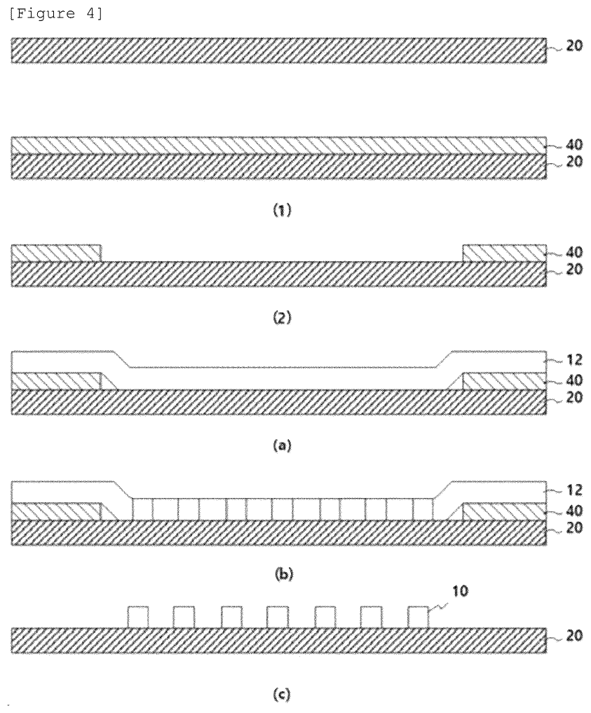

In addition, as shown in FIG. 4, the protrusion patch of the present invention may be characterized in that
before laminating the protrusion-forming sheet on one surface of the adhesive sheet in the step a), the method further includes the steps of:
1) adhering a release film 40 on one surface of the adhesive sheet 20; and
2) punching the center of the release film 40 to an area for arranging protrusions and removing the punched center release film 40 to form a hollow part,
wherein the protrusion-forming sheet 12 is laminated so as to cover the hollow part and the remaining release film,
punching multiple patterned protrusions into the protrusion-forming sheet in the step b) is performed on the protrusion-forming sheet arranged in the hollow part, and
when the protrusion-forming sheet 12 except for the protrusions patterned by the punching is removed from the adhesive sheet 20 in the step c), the release film 40 is also removed together.

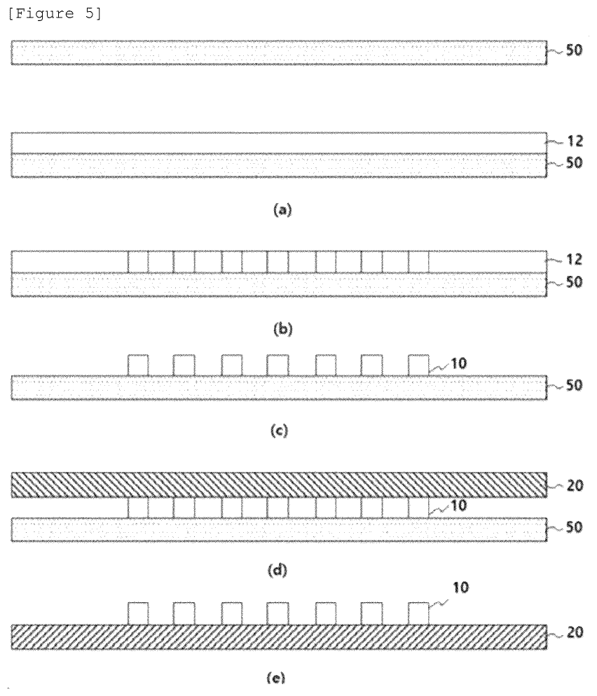

In another embodiment of the present invention, as shown in FIG. 5, the protrusion patch may be manufactured by including the steps of:
a) laminating a protrusion-forming sheet 12 on one surface of a first adhesive sheet 50;
b) punching multiple patterned protrusions 12 into the protrusion-forming sheet 12;
c) removing the protrusion-forming sheet 12 except for the protrusions 10 patterned by the punching from the first adhesive sheet 50;
d) laminating a second adhesive sheet 20 on the upper surface of the patterned protrusions 10; and
e) separating the second adhesive sheet 20 and the protrusions 10 adhered thereto from the first adhesive sheet 50.

The patterned protrusions contain a drug and a drug carrier, and
the protrusion-forming sheet may have a solvent content of 10% to 11% by weight.

In the manufacturing method, the adhesion of the second adhesive sheet to the protrusions may be greater than the adhesion of the first adhesive sheet to the protrusions.

In the step d), the second adhesive sheet may have a supporting substrate 30 further laminated on the other surface of the surface to which the protrusions are adhered.

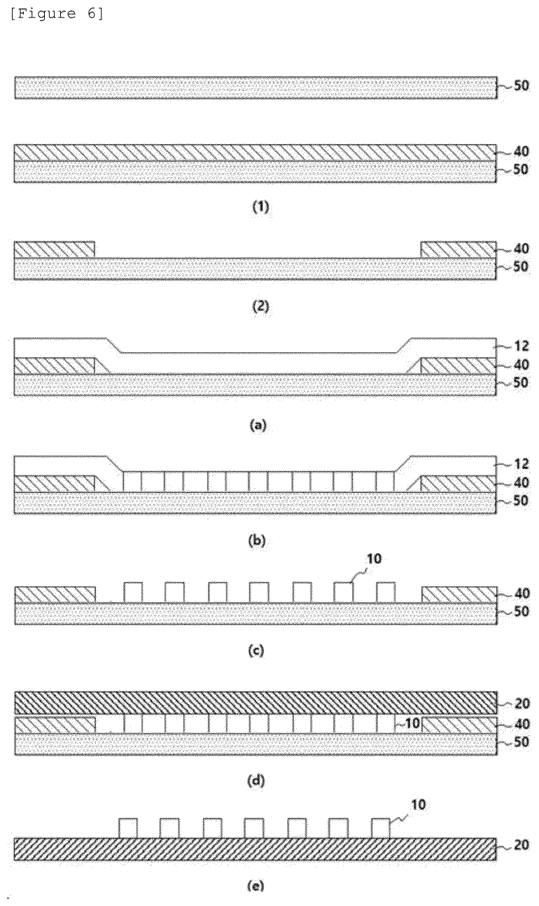

In one embodiment of the present invention, as shown in FIG. 6,
before laminating the protrusion-forming sheet 12 on one surface of the first adhesive sheet 50 in the step a), the method further includes the steps of:
1) adhering a release film 40 on one surface of the first adhesive sheet 50; and
2) punching the center of the release film 40 to an area for arranging protrusions and removing the punched center release film 40 to form a hollow part,
wherein the protrusion-forming sheet 12 is laminated so as to cover the hollow part and the remaining release film 40, and
punching multiple patterned protrusions 10 into the protrusion-forming sheet 12 in the step b) may be performed on the protrusion-forming sheet arranged in the hollow part.

Hereinafter, the present invention will be described in detail by way of examples in order to specifically describe the present invention. However, the examples according to the present invention may be modified in various different forms, and the scope of the present invention should not be construed as being limited to the examples described below. The examples of the present invention are provided to more fully describe the present invention to a person having ordinary skill in the art.

### Example 1: Manufacture of a Protrusion Patch

### (1) Manufacture of a Protrusion-Forming Sheet

A composition for a protrusion-forming sheet was manufactured by mixing 5% by weight of hyaluronic acid as a drug, 1.2% by weight of Hydrolyzed Collagen, 50% by weight of polyvinylpyrrolidone (PVP) as a drug carrier, 28.5% by weight of polyvinyl alcohol, 2.1% by weight of Pulluran, 1.2% by weight of PEG- 60 Hydrogenated Castor Oil, 1.5% by weight of sodium chloride, and 10.5% by weight of distilled water as a solvent. The composition was applied onto a polyethylene terephthalate (PET) liner (thickness of 75 μm) that had undergone silicone release treatment to manufacture a 40 μm thick protrusion-forming sheet.

### (2) Manufacture of an Adhesive Sheet

An adhesive component was obtained by mixing 43 parts by weight of Hydrogenated Poly (C6-20 Olefin), 18 parts by weight of styrene-isoprene copolymer, 28 parts by weight of carboxymethyl cellulose, and 11 parts by weight of mineral oil.

The adhesive component was applied onto a polyethylene terephthalate (PET) liner (thickness of 75 μm) that had undergone silicone release treatment so that the thickness become 300 μm after drying, and a moisture-permeable and waterproof polyurethane film was adhered thereto to manufacture an adhesive sheet.

### (3) Manufacture of a Protrusion Patch

The protrusion-forming sheet manufactured in the 1) was laminated on one surface of the adhesive layer of the adhesive sheet manufactured in the 2).

The protrusion-forming sheet was punched using a punching apparatus having a diameter of 1.25 mm.

The remaining protrusion-forming sheet except for the protrusions patterned by the punching from the adhesive layer was removed from the adhesive layer. Thereafter, a release paper was placed over the adhesive layer and the patterned protrusions, placed in packaging paper, and sealed to manufacture a protrusion patch.

### Example 2: Manufacture of a Protrusion Patch

A protrusion patch was manufactured in the same manner as in the Example 1, except that the amount of polyvinyl alcohol used in the manufacture of the protrusion-forming sheet in Example 1 was reduced to 28% by weight and the amount of distilled water used was increased to 11% by weight.

### Comparative Example 1: Manufacture of a Protrusion Patch

A protrusion patch was manufactured in the same manner as in the Example 1, except that the amount of polyvinyl alcohol used in the manufacture of the protrusion-forming sheet in Example 1 was increased to 29.7% by weight and the amount of distilled water used was reduced to 9.3% by weight.

### Comparative Example 2: Manufacture of a Protrusion Patch

A protrusion patch was manufactured in the same manner as in the Example 1, except that the amount of polyvinyl alcohol used in the manufacture of the protrusion-forming sheet in Example 1 was reduced to 27.8% by weight and the amount of distilled water used was increased to 11.2% by weight.

### Test Example 1: Quality Evaluation of Protrusion Patches

The shapes of the protrusion patches manufactured in the examples and comparative examples were photographed with a camera and evaluated.

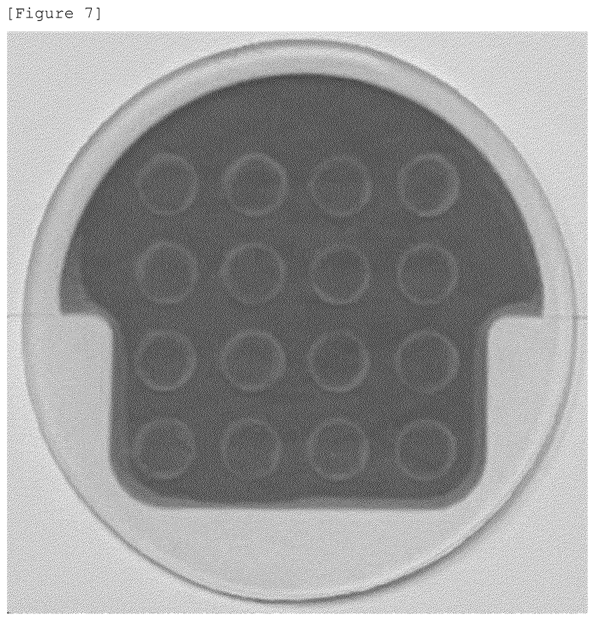

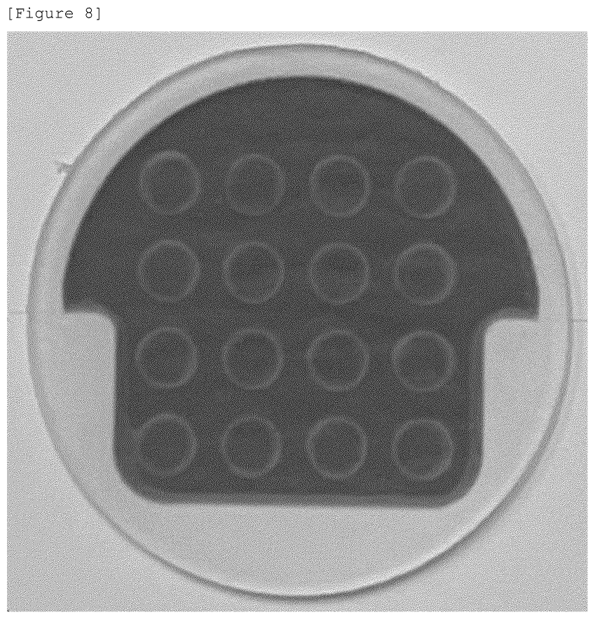

The evaluation results are shown in FIGS. 7 to 10. FIG. 7 is a photograph of the protrusion patch of Example 1 of the present invention, and FIG. 8 is a photograph of the protrusion patch of Example 2 of the present invention. As confirmed in the FIGS. 7 and 8, in the case of the protrusion patches of Examples 1 and 2 of the present invention, it can be seen that excellent quality protrusion patches were manufactured.

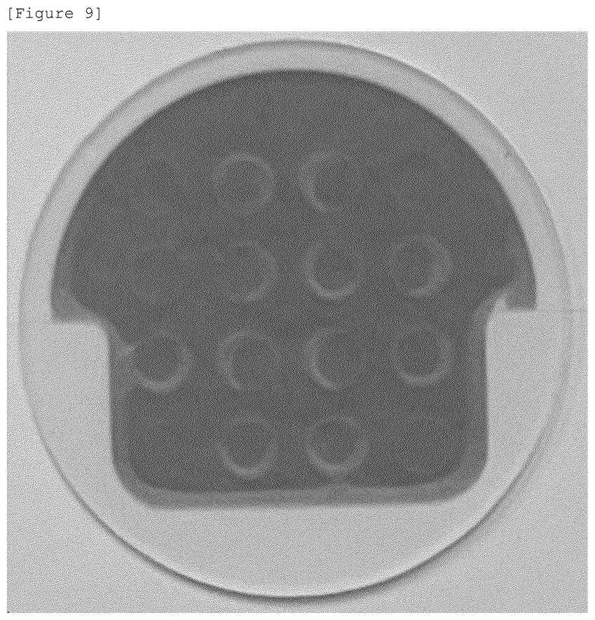

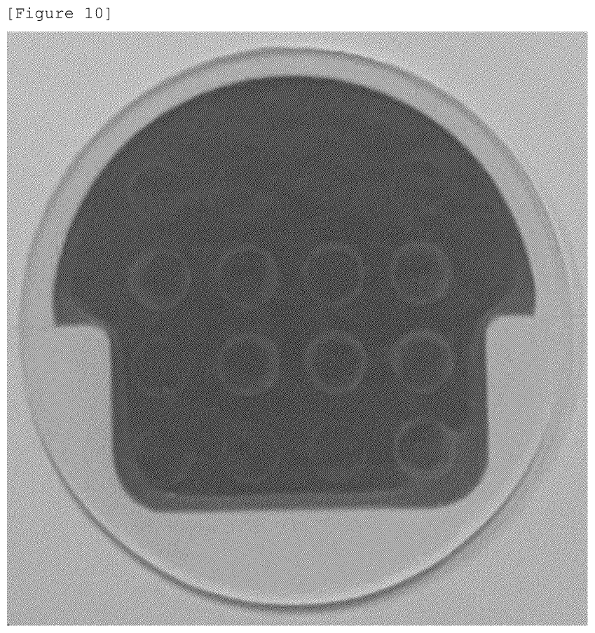

Meanwhile, FIG. 9 is a photograph of the protrusion patch of Comparative Example 1, and FIG. 10 is a photograph of the protrusion patch of Comparative Example 2. As confirmed in the FIGS. 9 and 10, in the case of the protrusion patches of Comparative Examples 1 and 2, it can be seen that the protrusions were detached from the adhesive layer when punched to result in the manufacture of defective products.

Therefore, it can be seen from the above experiment that the protrusions should contain a solvent in the range of 10% to 11% by weight, and if the solvent content is outside the above range, the protrusions are not fixed to the adhesive layer and are detached therefrom when punched to result in the manufacture of defective products.

Likewise, the protrusion-forming sheet should also contain solvent in the range of 10% to 11% by weight.

Although the preferred examples of the present invention have been described in detail above, the scope of a right for the present invention is not limited thereto, and various modifications and improvements made by those skilled in the art using the basic concept of the present invention defined in the following claims also fall within the scope of a right for the present invention.

### DESCRIPTION OF SYMBOLS

10: Protrusion 12: Protrusion-forming sheet
20: Adhesive layer (second adhesive layer) 30: supporting substrate
40: Release film 50: First adhesive layer
100: Protrusion patch

1. A protrusion patch, comprising:
an adhesive layer; and
multiple patterned protrusions spaced apart from each other and attached to one surface of the adhesive layer,
wherein the patterned protrusions contain a drug and a drug carrier, and
the protrusions have a solvent content of 10% to 11% by weight.

2. The protrusion patch according to claim 1, wherein the solvent is one or more selected from the group consisting of water, lower alcohol and glycerin.

3. The protrusion patch according to claim 1, wherein the patterned protrusions have a maximum width of 0.1 mm to 50 mm and a height of 0.01 mm to 1 mm.

4. The protrusion patch according to claim 1, wherein the protrusions have a shape of a cylinder, an elliptical column or a polygonal column.

5. The protrusion patch according to claim 1, wherein the drug is a drug for transdermal administration.

6. The protrusion patch according to claim 5, wherein the drug is hyaluronic acid.

7. The protrusion patch according to claim 1, wherein the drug carrier is one alone or a combination of two or more selected from the group consisting of hyaluronic acid, polyvinylpyrrolidone (PVP), polyvinyl alcohol (PVA), sodium carboxymethyl cellulose (NaCMC), poloxamer, carbomer, hypromellose, hydroxypropyl cellulose (HPC), hydroxyethyl cellulose (HEC), sodium alginate, saccharides, glycerin, propylene glycol, polyethylene glycol 400 and sorbitol (SB).

8. The protrusion patch according to claim 1, wherein a supporting substrate is further provided on the other surface of the adhesive layer.

9. A method for manufacturing a protrusion patch, comprising the steps of:
a) laminating a protrusion-forming sheet on one surface of an adhesive sheet;
b) punching multiple patterned protrusions into the protrusion-forming sheet; and
c) removing the protrusion-forming sheet except for the protrusions patterned by the punching from the adhesive sheet,
wherein the patterned protrusions contain a drug and a drug carrier, and
the protrusion-forming sheet has a solvent content of 10% to 11% by weight.

10. The method for manufacturing the protrusion patch according to claim 9, wherein the protrusions have a shape of a cylinder, an elliptical column or a polygonal column.

11. The method for manufacturing the protrusion patch according to claim 9, wherein a supporting substrate is further attached to the other surface of the adhesive sheet before step a) or after step c).

12. The method for manufacturing the protrusion patch according to claim 9, wherein before laminating the protrusion-forming sheet on one surface of the adhesive sheet in the step a), the method further comprises the steps of:
1) adhering a release film on one surface of the adhesive sheet; and
2) punching the center of the release film to an area for arranging protrusions and removing the punched center release film to form a hollow part,
wherein the protrusion-forming sheet is laminated so as to cover the hollow part and the remaining release film,
the punching of multiple patterned protrusions into the protrusion-forming sheet in the step b) is performed on the protrusion-forming sheet arranged in the hollow part, and
when the protrusion-forming sheet except for the protrusions patterned by the punching is removed from the adhesive sheet in the step c), the release film is also removed together.

13. A method for manufacturing a protrusion patch, comprising the steps:
a) laminating a protrusion-forming sheet on one surface of a first adhesive sheet;
b) punching multiple patterned protrusions into the protrusion-forming sheet;
c) removing the protrusion-forming sheet except for the protrusions patterned by the punching from the first adhesive sheet,
d) laminating a second adhesive sheet on the upper surface of the patterned protrusions; and
e) separating the second adhesive sheet and the protrusions adhered thereto from the first adhesive sheet,
wherein the patterned protrusions contain a drug and a drug carrier, and
the protrusion-forming sheet has a solvent content of 10% to 11% by weight.

14. The method for manufacturing the protrusion patch according to claim 13, wherein the protrusions have a shape of a cylinder, an elliptical column or a polygonal column.

15. The method for manufacturing the protrusion patch according to claim 13, wherein the adhesion of the second adhesive sheet to the protrusions is greater than the adhesion of the first adhesive sheet to the protrusions.

16. The method for manufacturing the protrusion patch according to claim 13, wherein in the step d), the second adhesive sheet has a supporting substrate further laminated on the other surface of the surface to which the protrusions are adhered.

17. The method for manufacturing the protrusion patch according to claim 13, wherein before laminating the protrusion-forming sheet on one surface of the first adhesive sheet in the step a), the method further comprises the steps of:
1) adhering a release film on one surface of the first adhesive sheet; and
2) punching the center of the release film to an area for arranging protrusions and removing the punched center release film to form a hollow part,
wherein the protrusion-forming sheet is laminated so as to cover the hollow part and the remaining release film, and
the punching of multiple patterned protrusions into the protrusion-forming sheet in the step b) is performed on the protrusion-forming sheet arranged in the hollow part.
